(19) United States
(12) Patent Application Publication
(10) Pub. No.: US 2010/0061972 A1
(43) Pub. Date: Mar. 11, 2010

(54) Method of Treating Peyronie's Disease

(75) Inventor: Theodore Smith, Schwenksville, PA (US)

Correspondence Address: ELMORE PATENT LAW GROUP, PC, 515 Groton Road, Unit 1R, Westford, MA, 01886, US

(73) Assignee: Auxilium US Holdings,LLC, Malvem, PA (US)

(21) Appl. No.: 12/542,759

(22) Filed: Aug. 18, 2009

Related U.S. Application Data
(60) Provisional application No. 61/089,944, filed on Aug. 19, 2008.

(51) Int. Cl. A61K 38/48; A61P 15/00; G06F 19/00; A61B 5/00

(52) U.S. Cl. 424/94.67; 702/19; 600/300

(57) ABSTRACT

The invention is directed to methods of treating patients suffering from Peyronie's disease, methods of identifying patients suffering from Peyronie's disease and a computer program for identifying patients suffering from Peyronie's disease.

### RELATED APPLICATION

This application claims the benefit of U.S. Provisional Application No. 61/089,944, filed on Aug. 19, 2008. The entire teachings of the above application(s) are incorporated herein by reference.

### BACKGROUND OF THE INVENTION

Peyronie's disease is an idiopathic condition resulting in penile deformity and disability as the result of scarring and contracture within the tunica albugines of the corpora cavernosa. The scarring takes the form of plaques or masses of dense fibrous tissue and results in a curvature of the penis during erection. Peyronie's disease may result in pain or discomfort during erection, ejaculation and/or intercourse. The etiology of Peyronie's disease is not known, however, several factors have been associated with a greater likelihood of Peyronie's disease including a history of Dupuytren's disease, diabetes, use of beta-blocker therapy and hypertension (Sommer et al. 2002, International Journal of Impotence Research, 14(5): 379-383).

One-third of patients suffering from Peyronie's disease improve without treatment within about 18 months. The remainder of patients require surgical or pharmacologic treatment. Drugs that may be used in the treatment of Peyronie's disease include vitamin E, colchicine, verapamil and calcium channel blockers. In addition, U.S. Pat. No. 6,022,539 describes the use of the enzyme collagenase for the treatment of Peyronie's disease.

Since Peyronie's disease is associated with symptoms that may be considered to be embarrassing, patients are often reluctant to discuss their symptoms with their physicians. Consequently, patients suffering from Peyronie's disease often go undiagnosed and untreated. It would be advantageous to develop a method for identifying patients suffering from the symptoms and adverse effects of Peyronie's disease and that would allow patients to communicate their symptoms with candor and without embarrassment.

### SUMMARY OF THE INVENTION

The invention is directed to methods of treating patients suffering from Peyronie's disease, methods of identifying patients suffering from Peyronie's disease, a computer program for identifying patients suffering from Peyronie's disease and a computer program for determining the suitability of collagenase therapy.

In one embodiment, the invention is directed to a method of treating patients suffering from Peyronie's disease wherein the method comprises:
a. gathering an information set from a patient suspected of suffering from Peyronie's disease wherein said information set comprises a subjective parameter relating to the presence or severity of pain or discomfort during the patient's most recent experience of intercourse, erection or ejaculation, wherein the information set is gathered using a written questionnaire;
b. comparing the information set gathered from the patient suspected of suffering from Peyronie's disease with a standardized symptom profile of a patient suffering from Peyronie's disease;
c. determining whether the patient is suffering from Peyronie's disease; and
d. administering collagenase to said patient suffering from Peyronie's disease.

In another embodiment, the invention is a computer program embodied on a computer readable medium for identifying a patient suffering from Peyronie's disease comprising:
a. a code segment for providing at least two questions assessing the presence or severity of pain or discomfort during the patient's most recent experience of intercourse, erection or ejaculation; and
b. a code segment for receiving answers to the questions.

The invention additionally encompasses methods of diagnosing Peyronie's disease and to methods of determining the suitability of collagenase therapy.

### DETAILED DESCRIPTION OF THE INVENTION

A description of preferred embodiments of the invention follows.

The words “a” and “an” are meant to encompass one or more unless otherwise specified.

In one embodiment, the invention is directed to a method of treating Peyronie's disease by administering collagenase, a method for determining the suitability of collagenase therapy, and/or to a method of diagnosing Peyronie's disease in a patient suspected of suffering therefrom comprising the following steps:
a. gathering an information set from a patient suspected of suffering from Peyronie's disease wherein said information set comprises a subjective parameter relating to the presence or severity of pain or discomfort during the patient's most recent experience of intercourse, erection or ejaculation, wherein the information set is gathered using a written questionnaire;
b. comparing the information set gathered from the patient suspected of suffering from Peyronie's disease with a standardized symptom profile of a patient suffering from Peyronie's disease; and
c. determining whether the patient is suffering from Peyronie's disease or whether the patient is likely to benefit from collagenase.

In additional embodiments, the inventive methods further comprise administering collagenase to said patient suffering from Peyronie's disease.

“Treating” or “treatment” includes preventing or delaying the onset of the symptoms, complications, or biochemical indicia of a disease, alleviating or ameliorating the symptoms or arresting or inhibiting further development of the disease, condition, or disorder.

A subjective parameter is the patient's subjective impression with respect to a symptom of Peyronie's disease and includes the experience of pain or discomfort or the severity of that pain or discomfort during intercourse, erection or ejaculation.

An information set comprises data provided by the patient or responses to questions asked of the patient either verbally or in writing. According to the present invention, the information set is gathered using a written questionnaire, such as a confidential written questionnaire. In one embodiment, the information set comprises subjective parameters relating to the presence and severity of pain before an erection (or in a non-erect penis) and/or during at least one of intercourse, ejaculation or erection (whether it be spontaneous or in response to stimuli). In another embodiment, the information set comprises subjective parameters relating to the presence and severity of pain before an erection and/or during at least two of erection, intercourse, or ejaculation (e.g. intercourse and ejaculation; erection and intercourse; and erection and ejaculation, e.g., as a result of masturbation). In another embodiment, the information set comprises subjective parameters relating to the presence and severity of pain before an erection and/or during all three of intercourse, ejaculation or erection.

In other embodiments, the information set can further comprise one or more different sets of responses to questions such as those relating to the experience of pain or discomfort during intercourse, erection or ejaculation, those relating to the level of pain or discomfort experienced and those relating to the patient's distress or negative thoughts or feeling regarding the experience of pain, discomfort or physical symptoms. In one embodiment, the information set comprises responses to a first set of questions directed to the experience of pain or discomfort during intercourse, erection or ejaculation and a second set of questions directed to the level of pain or discomfort experienced. The level of pain or discomfort experienced can be described using a numerical scale. The presence of pain or discomfort experienced can also be described qualitatively. In an additional embodiment, the information set further comprises a third set of questions directed to the patient's distress or negative thoughts or feelings associated with the experience of pain or discomfort. Such questions include those directed to the effect of the pain, discomfort or physical symptoms on the patient's mood, self-esteem and/or lifestyle.

Physical symptoms of Peyronie's disease include, but are not limited to, curvature of the penis during erection and the formation of a fibrous plaque.

A standardized symptom profile of a patient suffering from Peyronie's disease is a data set comprising a group of symptoms of Peyronie's disease experienced by persons suffering from the disease. As used herein, a “group” is meant to signify at least two.

The standardized symptom profile is typically compiled by identifying at least one symptom selected from the group consisting of pain or discomfort during intercourse, ejaculation or erection, physical symptoms, negative thoughts or feelings associated with having pain or discomfort, and combinations thereof wherein the symptoms are associated with Peyronie's disease. In one embodiment, the standardized symptom profile includes at least 2, 3, 4, 5, 6, 7, 8, 9, 10, 11, 12, 13, 14 or more symptoms.

In some embodiments, the information set gathered from the patient is compared to the standardized symptom profile and the patient is identified as suffering from Peyronie's disease when the patient is identified as suffering from at least 1, 2, 3, 4, 5, 6, 7, 8, 9, 10, or more symptoms included in the standardized symptom profile.

In addition to comparing the information set gathered from the patient in order to determine whether the patient is suffering from Peyronie's disease, the patient can also be physically examined by a medical practitioner such as a urologist. In one embodiment, the information set gathering is preceded by physical examination. In another embodiment, the information set is followed by physical examination. Information gathered from the physical examination as well as that from the information set can be used to identify a patient as suffering from Peyronie's disease or to determine the suitability of collagenase treatment.

Collagenase is an enzyme that has the specific ability to digest collagen. It is derived commercially from fermentation by Clostridium histolyticum, and is purified by a chromatographic technique. It is available in several levels of purity, containing from substantial to essentially zero amounts of other proteinases.

The use of collagenase for the treatment of patients suffering from Peyronie's disease has been described in the literature, for example, in U.S. Pat. No. 6,022,539, the contents of which are incorporated by reference herein. Methods of administering collagenase include injection into a fibrous Peyronie's plaque. In some embodiments, the penis is immobilized immediately after injection. Methods of immobilization include wrapping with sufficient gauze bandage and in such a way as to form a bulky dressing, and/or by the patient's donning an athletic supporter. The immobilization can be continued for several hours, for example from about 4 to about 12 hours. Typically, if the injection takes place during the early part of the afternoon, the dressing/support is removed at bedtime.

The amount and concentration of collagenase used is that amount and concentration which is effective to soften and/or rupture the plaque. In one embodiment, a collagenase solution as described in U.S. Ser. No. 11/335,157, filed on Jan. 19, 2006, which is incorporated herein by reference is administered. The collagenase is injected into the Peyronie's plaque to provide a total amount of at least about 20,000 ABC units of collagenase in a pharmaceutically acceptable carrier in a concentration of about 20,000 to about 40,000 ABC units per ml. The total amount may be applied by way of one or more injections. The maximum cumulative total dosage can be limited to about 60,000 ABC units. In a preferred embodiment, 0.58 mg of highly purified Clostridial collagenase having a 1:1 mass ratio of the colG and colH is administered.

The invention may additionally encompass a computer program embodied on a computer readable medium for identifying a patient suffering from Peyronie's disease or determining the suitability of collagenase therapy comprising:
a. a code segment for providing at least two questions assessing the presence or severity of pain or discomfort during the patient's most recent experience of intercourse, erection or ejaculation; and
b. a code segment for receiving answers to the questions.

In some embodiments, the computer program further comprises:
c. a code segment for identifying a patient suffering from Peyronie's disease based on the patient's answers to the questions.

In another embodiment, a computer program may have a code segment for representing the standardized symptom profile and a code segment for representing the answers to a questionnaire (whether administered orally, in writing, or by another aspect of a computer program), wherein the computer program is adapted to compare the answers to the standardized symptom profile and thereby identify a patient suffering from Peyronie's disease.

In an additional embodiment, the invention is directed to a written questionnaire comprising at least three questions directed to the experience of pain or discomfort during intercourse, erection or ejaculation, the level of pain or discomfort experienced or to the patient's distress or negative thoughts or feeling regarding the experience of pain and discomfort. In another embodiment, the questionnaire comprises a first set of questions directed to the experience of pain or discomfort during intercourse, erection or ejaculation and a second set of questions directed to the level of pain or discomfort experienced. In a further embodiment, the written questionnaire further comprises a third set of questions directed to the patient's distress or negative thoughts or feelings associated with the experience of pain or discomfort. In yet another embodiment, the written questionnaire comprises questions substantially as shown in Tables 1 to 3.

TABLE 1
Thinking of your most recent sexual experience, please indicate how
much you agree or disagree with each of the following statements.

I am afraid of damaging my penis while having vaginal intercourse.
I am afraid my penis might bend or collapse along the base while having
vaginal intercourse.
I am afraid I might feel pain or discomfort in my penis while having
vaginal intercourse.
I am afraid I might have trouble inserting my erect penis into my partner's
vagina.
When I begin to ejaculate, I am afraid my semen might be all stopped up
inside for a few seconds.
I feel that my erect penis is not large enough to fill my partner's vagina.
I have had to give up having vaginal intercourse in certain positions that I
used to enjoy.
When having vaginal intercourse, some positions I used to enjoy are now
awkward for me.
When having vaginal intercourse, some positions I used to enjoy are now
uncomfortable for me.

TABLE 2
Thinking of your last erection or ejaculation, or the last time you
had vaginal intercourse, please indicate the level of pain or
discomfort you felt.

In the last 24 hours, how much pain or discomfort have you felt in your
penis when it was NOT erect?
Thinking about the last time you were erect, how much pain or discomfort
did you feel in your penis when it was erect.
Thinking about the last time you had vaginal intercourse, how much pain
or discomfort did you feel in your penis when having vaginal
intercourse?
Thinking about the last time you ejaculated, how much pain or discomfort
did you feel in your penis while ejaculating?

TABLE 3
Please answer the following questions regarding problems you
may have experienced.

Thinking about the last time you had an erection, how bothered were you
by any pain or discomfort you may have felt in your erect penis?
Thinking about the last time you ejaculated, how bothered were you by
any problem you may have had ejaculating?
Thinking about the last time you looked at your erect penis, how bothered
were you by the way your penis looked?
Do you have a physical problem that makes having vaginal intercourse
difficult or impossible?
Thinking of the last time you had or tried to have vaginal intercourse, how
bothered were you by any physical problem you may have had?
Are you having vaginal intercourse less often than you used to due to your
physical problem?
How bothered are you with having vaginal intercourse less often?

While this invention has been particularly shown and described with references to preferred embodiments thereof, it will be understood by those skilled in the art that various changes in form and details may be made therein without departing from the scope of the invention encompassed by the appended claims.

What is claimed is:

1. A method for the treatment of Peyronie's disease in a patient suffering therefrom by administering collagenase to said patient, wherein the method comprises:
a. gathering an information set from a patient suspected of suffering from Peyronie's disease wherein said information set comprises a subjective parameter relating to the presence or severity of pain or discomfort during the patient's most recent experience of intercourse, erection or ejaculation, wherein the information set is gathered using a written questionnaire;
b. comparing the information set gathered from the patient suspected of suffering from Peyronie's disease with a standardized symptom profile of a patient suffering from Peyronie's disease;
c. determining whether the patient is suffering from Peyronie's disease; and
d. administering collagenase to said patient suffering from Peyronie's disease.

2. The method of claim 1, wherein the information set further comprises a subjective parameter to assess the presence of a physical symptom.

3. The method of claim 1, wherein the information set comprises a subjective parameter relating to the presence or severity of pain or discomfort in a non-erect penis and/or during intercourse.

4. The method of claim 3, wherein the information set comprises subjective parameters relating the presence and severity of pain or discomfort.

5. The method of claim 1, wherein the information set comprises a subjective parameter relating to the presence or severity of pain or discomfort during erection.

6. The method of claim 5, wherein the information set comprises subjective parameters relating the presence and severity of pain or discomfort.

7. The method of claim 1, wherein the information set comprises a subjective parameter relating to the presence or severity of pain or discomfort during ejaculation.

8. The method of claim 7, wherein the information set comprises subjective parameters relating the presence and severity of pain or discomfort.

9. The method of claim 1, wherein the information set comprises subjective parameters relating to the presence or severity of pain or discomfort during intercourse and erection.

10. The method of claim 1, wherein the information set comprises subjective parameters relating to the presence or severity of pain or discomfort during intercourse, erection and ejaculation.

11. The method of claim 1, wherein the information set further comprises a subjective parameter relating to the fear of experiencing pain or discomfort during intercourse.

12. The method of claim 1, wherein the information set is gathered using a confidential written questionnaire.

13. The method of claim 1, wherein the information set comprises responses to a first set of questions directed to experience of pain or discomfort during intercourse, erection or ejaculation and a second set of questions directed to the level of pain or discomfort experienced.

14. The method of claim 13 comprising a third set of questions directed at assessing the patient's distress regarding the experience of pain or discomfort.

15. The method of claim 1, wherein collagenase is administered at a dose of at least about 20,000 ABC units.

16. The method of claim 1, wherein collagenase in a pharmaceutically acceptable carrier at a concentration of about 20,000 to about 40,000 ABC units per ml.

17. The method of claim 1, wherein the collagenase is administered by injection.

18. The method of claim 1, wherein said information set gathering is followed by physical examination by a medical practitioner.

19. The method of claim 1, wherein said information set gathering is preceded by a physical examination by a medical practitioner.

20. The method of claim 17, wherein the medical practitioner is an urologist.

21. A computer program embodied on a computer readable medium for identifying a patient suffering from Peyronie's disease comprising:
a. a code segment for providing at least two questions assessing presence or severity of pain or discomfort during the patient's most recent experience of intercourse, erection or ejaculation; and
b. a code segment for receiving answers to the questions.

22. The computer program of claim 21 further comprising:
c. a code segment for identifying a patient suffering from Peyronie's disease based on the patient's answers to the questions.

23. A method for determining the suitability of collagenase therapy comprising identifying a patient suspected of suffering from Peyronie's disease wherein said patient is identified by:
a. gathering an information set from a patient suspected of suffering from Peyronie's disease wherein said information set comprises a subjective parameter relating to the presence or severity of pain or discomfort during the patient's most recent experience of intercourse, erection or ejaculation, wherein the information set is gathered using a written questionnaire;
b. comparing the information set gathered from the patient suspected of suffering from Peyronie's disease with a standardized symptom profile of a patient suffering from Peyronie's disease; and
c. determining whether the patient is likely to benefit from collagenase therapy.

24. A method of diagnosing Peyronie's disease in a patient suspected of suffering therefrom comprising:
a. gathering an information set from a patient suspected of suffering from Peyronie's disease wherein said information set comprises a subjective parameter relating to the presence or severity of pain or discomfort during the patient's most recent experience of intercourse, erection or ejaculation, wherein the information set is gathered using a written questionnaire;
b. comparing the information set gathered from the patient suspected of suffering from Peyronie's disease with a standardized symptom profile of a patient suffering from Peyronie's disease; and
c. determining whether the patient is suffering from Peyronie's disease.
